(19) United States
(12) Patent Application Publication
(10) Pub. No.: US 2024/0366199 A1
(43) Pub. Date: Nov. 7, 2024

(54) FINE NEEDLE ASPIRATION (FNA) SYRINGE ASSEMBLIES AND METHODS OF USING THE SAME

(71) Applicant: SEAN LYMAN, CHARLOTTESVILLE, VA (US)

(72) Inventor: SEAN LYMAN, CHARLOTTESVILLE, VA (US)

(21) Appl. No.: 18/774,460

(22) Filed: Jul. 16, 2024

Related U.S. Application Data
(62) Division of application No. 18/110,457, filed on Feb. 16, 2023.
(60) Provisional application No. 63/405,650, filed on Sep. 12, 2022.

(51) Int. Cl. A61B 10/02

(52) U.S. Cl. CPC A61B 10/0283; A61B 2010/0208

(57) ABSTRACT

A syringe assembly includes a syringe casing defining an aspiration cavity therein and including a barrel having opposite first and second ends, and a plunger movable within the barrel to define a volume of the aspiration cavity. The syringe assembly also includes a hollow needle having a distal end configured for piercing tissue, and a proximal end detachably fixed to the first end of the syringe casing, the hollow needle in fluid communication with the aspiration cavity. The syringe assembly further includes a stylet having a distal portion extending within the needle and a proximal portion extending within the syringe casing, an extraction mechanism coupled to the proximal portion of the stylet, and a trigger at least partially exposed externally of the aspiration cavity of the syringe casing. The trigger when activated causes the extraction mechanism to extract the stylet from the needle.

### CROSS-REFERENCE TO RELATED APPLICATION

This is a Divisional of U.S. Non-Provisional patent application Ser. No. 18/110,457, filed Feb. 16, 2023, and a claim of priority is made to U.S. Provisional Patent Application No. 63,405,650, filed Sep. 12, 2022, the disclosure of which is incorporated by reference herein in its entirety.

### BACKGROUND

The inventive concepts generally relate to fine needle aspiration (FNA), and more particularly, the inventive concepts relate to FNA syringe and needle assemblies and to methods of using FNA syringe and needle assemblies.

Fine needle aspiration (FNA) is a common procedure used in the diagnosis of multiple medical problems, most commonly diagnosis of cancer. The procedure is performed using a fine gauge aspiration needle (usually 20-25 gauge) with a stylet. In particular, a hollow needle is typically configured with an engagement hub at one end, and a stylet may also be configured with an engagement hub at one end. The engagement hub of the needle is configured for coupling to and de-coupling from the engagement hub of the stylet, and also for coupling to and de-coupling from the hub of a syringe. In an engaged state, the stylet is inserted into the hollow needle with their engagement hubs coupled to one another. In some cases the stylet is devoid of a hub, in which case it may be held in place within the hollow needle by friction.

The FNA procedure may be carried out by inserting the hollow needle with the stylet into an abnormal soft tissue structure under ultrasound guidance, removing the stylet, and then attaching the syringe to the needle with the stylet removed. The stylet is intended to prevent or inhibit non-target cells from blocking the aspiration needle during insertion. Negative pressure is then applied to the syringe while moving the needle within the lesion, packing a lumen of the needle with cells which are then expelled onto slides for pathologic evaluation. Typically the negative pressure is achieved by slowly withdrawing a plunger of the syringe.

One difficulty with the conventional method of obtaining fine needle aspirates is that the needle is held in one hand with the ultrasound probe in the other to insert the needle, but once the needle is in place the operator needs to put down the ultrasound probe in order to remove the stylet and attach the syringe. Then the probe must be picked up again and the needle and lesion relocated before the aspirate can be obtained.

Particularly in the case of very small lesions, this can add time and frustration to the procedure. In addition, the needle tip can be displaced from the lesion during removal of the stylet and attachment of the syringe leading to a suboptimal specimen.

### SUMMARY

According to an aspect of the inventive concepts, a syringe assembly is provided that includes a syringe casing defining an aspiration cavity therein and including a barrel having opposite first and second ends, and a plunger movable within the barrel to define a volume of the aspiration cavity. The syringe assembly also includes a hollow needle having a distal end configured for piercing tissue, and a proximal end detachably fixed to the first end of the syringe casing, the hollow needle in fluid communication with the aspiration cavity. The syringe assembly further includes a stylet having a distal portion extending within the needle and a proximal portion extending within the syringe casing, an extraction mechanism coupled to the proximal portion of the stylet, and a trigger at least partially exposed externally of the aspiration cavity of the syringe casing. The trigger when activated causes the extraction mechanism to extract the stylet from the needle.

The extraction mechanism may be configured to store potential energy, and the trigger may be configured to release the stored potential energy of the extraction mechanism to extract the stylet from the hollow needle into the syringe. The potential energy may be at least one of mechanical or electrical energy.

The extraction mechanism may be located within the barrel of the syringe casing.

The extraction mechanism may be located within the plunger.

The extraction mechanism may include an extraction chamber located within the barrel of the syringe casing, and the extraction chamber may be configured to house the stylet when extracted from the hollow needle into the syringe. The extraction mechanism may be a spring-loaded device and may further include a spring located within the extraction chamber and coupled to the proximal portion of the stylet, where the spring is configured to extract the stylet from the hollow needle into the extraction chamber when the trigger is activated. The spring may be helical and extend coaxially within the extraction chamber, where the trigger when activated releases a stored potential energy of the spring to draw the stylet into the syringe. The spring may be an extension spring. The spring may be a compression spring.

The extraction mechanism may include an extraction chamber extending coaxially within the barrel of the syringe casing, where the extraction chamber is configured to house the stylet when extracted from the hollow needle. The plunger may extend around and be movable along a length of the extraction chamber. The plunger may include a plunger seal extending around the extraction chamber. The plunger seal may include an inner surface in contact with an outer wall of the extraction chamber, and an outer surface in contact with an inner wall of the barrel of the syringe casing.

The extraction mechanism may be at least partially located within the plunger, and the plunger may be hollow to define an extraction chamber therein, where the extraction chamber is configured to house the stylet when extracted from the hollow needle. The extraction mechanism may be a spring-loaded device and further include a spring located within the extraction chamber of the plunger and coupled to the proximal portion of the stylet, and the spring may be configured to extract the stylet from the hollow needle into the extraction chamber of the plunger when the trigger is activated. The spring may be helical and extend coaxially within the extraction chamber of the plunger, and the trigger when activated may release a stored potential energy of the spring to draw the stylet into the syringe. The spring may be an extension spring. The spring may be a compression spring. The plunger may include a plunger seal having an opening therein, where the stylet extends through the opening in the plunger seal.

According to another aspect of the inventive concepts, a plunger for a syringe assembly is provided. The plunger includes a hollow body configured for insertion into a barrel of a syringe casing of a syringe assembly, the hollow body defining an extraction chamber therein. The plunger further includes an extraction mechanism located within the extraction chamber of the hollow body, the extraction mechanism configured to extract a stylet of the syringe assembly into the extraction chamber when triggered, and a seal located at one end of the hollow body, the seal including an opening for extracting the stylet there through into the extraction chamber.

The trigger may be configured to release a stored potential energy of the extraction mechanism to extract the stylet into the extraction chamber when activated. The potential energy may be at least one of mechanical or electrical energy. The extraction mechanism may be a spring-loaded device and include a spring located within the extraction chamber of the hollow body, and the spring may be configured to extract the stylet into the extraction chamber of the hollow body when the trigger is activated. The spring may be helical and extend coaxially within the extraction chamber of the hollow body, and the trigger when activated may release a stored potential energy of the spring to draw the stylet into the extraction chamber of the hollow body. The spring may an extension spring. The spring may be a compression spring.

According to yet another aspect of the inventive concepts, a syringe casing is provided that includes a syringe casing body including a barrel having opposite first and second ends, wherein the first end of the barrel is configured to receive a plunger. The syringe casing further includes a hub located at the second end of the syringe casing body, the hub configured for attaching a hollow needle to the syringe casing body and including an opening configured to receive a proximal portion of stylet into the syringe casing body. The syringe casing still further includes an extraction mechanism located within the barrel of the syringe casing, and a trigger at least partially exposed externally of the syringe casing, where the trigger when activated causes the extraction mechanism to extract the stylet from the hollow needle into the barrel of the syringe casing.

The extraction mechanism may include an extraction chamber located in the barrel of the syringe casing body. The trigger may be configured when activated to release a stored potential energy of the extraction mechanism to extract the stylet into the extraction chamber. The potential energy may be at least one of mechanical or electrical or magnetic energy. The extraction mechanism may be a spring-loaded device and includes a spring located within the extraction chamber, and the spring may be configured to extract the stylet into the extraction chamber when the trigger is activated. The spring may be helical and extends coaxially within the extraction chamber, and the trigger when activated may release a stored potential energy of the spring to draw the stylet into the extraction chamber. The spring may be an extension spring. The spring may be a compression spring.

According to still another aspect of the inventive concepts, a pre-packaged syringe assembly is provided that includes the afore-described syringe assembly and an outer package containing the syringe assembly.

The pre-package syringe assembly may further include a protective sheath covering the hollow needle within the outer package.

According to another aspect of the inventive concepts, a method of fine need aspiration (FNA) is provided that includes providing the afore-described syringe assembly. The method further includes using one hand to hold the syringe assembly to insert the hollow needle into tissue while using another hand to hold an ultrasound probe to assist in positioning the needle in the tissue, activating the trigger using the one hand to cause the extraction mechanism to fully extract the stylet from the hollow needle into the extraction chamber while continuing to hold the ultrasound probe in the other hand, and moving the plunger using the one hand to create negative pressure within the aspiration cavity while continuing to hold the ultrasound probe in the other hand, wherein the negative pressure causes tissue sample to be drawn into the needle.

The method may further include extracting the hollow needle from the tissue, separating the hollow needle from the syringe casing, extracting the tissue sample from the hollow needle, and disposing of the hollow needle and syringe casing.

The method may further include extracting the tissue sample from the syringe casing prior to disposing of the syringe casing.

### BRIEF DESCRIPTION OF THE DRAWINGS

The various aspects and features of the inventive concepts will become readily apparent from the detailed description that follows, with reference to the accompanying drawings, in which:

FIG. 1 is a perspective view of a syringe casing according to one or more embodiments of the inventive concepts;

FIG. 2 is a perspective view of a plunger according to one or more embodiments of the inventive concepts;

FIGS. 3A, 3B and 4 are perspective views of a hollow needle and stylet according to one or more embodiments of the inventive concepts;

FIGS. 5A and 5B are perspective views of a syringe assembly according to one or more embodiments of the inventive concepts;

FIGS. 6A, 6B, 6C, 7A and 7B are perspective views of a syringe casing including a stylet extraction mechanism according to one or more embodiments of the inventive concepts;

FIG. 8 is a perspective view of a plunger including a stylet extraction mechanism according to one or more embodiments of the inventive concepts;

FIG. 9 is a cross-sectional view of a plunger including a stylet extraction mechanism according to one or more embodiments of the inventive concepts;

FIGS. 10A, 10B, 10C and 10D are perspective views of a plunger including a stylet extraction mechanism according to one or more embodiments of the inventive concepts;

FIGS. 11A and 11B are side views for reference in describing a spring clamp for retaining a stylet according to one or more embodiments of the inventive concepts;

FIGS. 12A, 12B, 12C and 12D are perspective views for reference in describing a stylet extraction trigger one or more embodiments of the inventive concepts; and

FIG. 13 is a flowchart for reference in describing a method of Fine Needle Aspiration (FNA) according to one or more embodiments of the inventive concepts . . .

### DETAILED DESCRIPTION

The inventive concepts will now be described in detailed by way of exemplary embodiments of the inventive concepts. Like elements and components are given like reference numbers throughout the drawings. Further, the drawings are not necessarily drawn to scale, and relative dimensions may be exaggerated for clarity of the description.

As will be described below, the inventive concepts present an integrated device which combines an echogenic FNA needle with a retractable stylet and an aspiration syringe capable of operation with one hand. The device would permit operators to access the lesion, retract the stylet, and obtain an aspirate under suction under continuous sonographic control. This would decrease procedure time, improve sample yield, and increase patient safety by keeping the needle visible to the operator at all times.

In an embodiment, the device is a syringe with an extraction chamber which contains a mechanism to retract the stylet. Retraction may be actuated, for example, by a button or trigger on the syringe. In some embodiments, a syringe plunger is designed to surround this extraction chamber and would be operated in a manner familiar to those who perform FNA. In other embodiments, the extraction chamber is contained within the plunger itself.

The device may ship assembled. The operator would insert the needle into the lesion, press the button to retract the stylet, and then aspirate as usual to obtain the sample. The entire device would then be passed to the pathology technician, who would then remove the needle and create smears for pathologic evaluation. Typically a small amount of preservative is then used to rinse the syringe to remove any sample that was aspirated into the syringe during sample collection.

As will be explained in detail below, the syringe assembly of some embodiments of the inventive concepts generally includes a syringe casing, a plunger, a hollow needle, a stylet, and a stylet extraction mechanism.

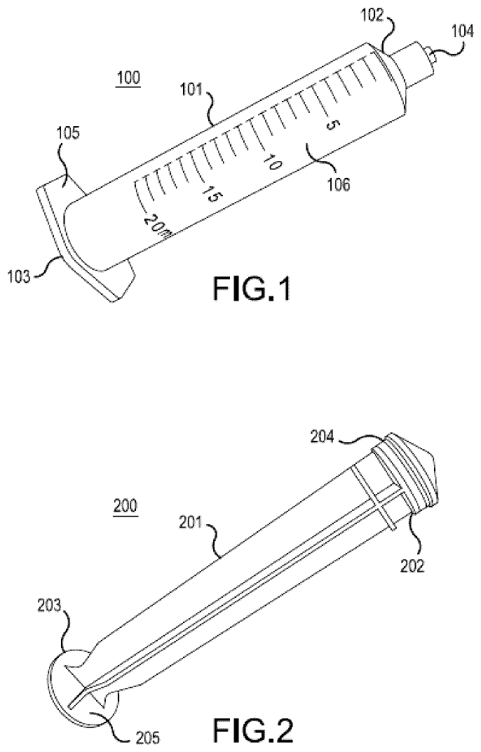

FIG. 1 is a perspective view of an example of a syringe casing 100. The syringe casing 100 of this example includes a barrel 101 defining an aspiration cavity 106 therein. The barrel 101 may be tubular (i.e., having a circular cross-section along its length) and include opposite first and second ends 102 and 103. In addition, the barrel 101 may include calibration markings (such as those shown in FIG. 1), and may be made of a transparent or semi-transparent material. At the first end 102 of the barrel 101, the syringe casing 100 may include an adaptor or hub 104 for connection to a needle assembly (described below). At the second end 103 of the barrel 101, the syringe casing 100 may include one or more flanges 105 to assist in manual manipulation of the syringe assembly during use. The syringe casing 100 is presented here as an example only, and embodiments of the inventive concepts are not limited to the particularities of the syringe casing 100.

FIG. 2 is a perspective view of an example of a plunger 200. The plunger 200 of this example includes a plunger body 201 having opposite first and second ends 202 and 203. The plunger body 201 is configured for insertion into the syringe casing 100, and is movable within the barrel 101 of the syringe casing 100 to define a volume of the aspiration cavity 106. At the first end 202 of the plunger body 201 is a plunger seal 204. The plunger seal 204 is configured to provide an air-tight seal at the inner surface of the barrel 101. In this manner, positive air pressure may be exerted in the aspiration cavity 106 by moving the plunger towards the first end 102 of the barrel 101, and negative pressure may be exerted in the aspiration cavity 106 by extracting the plunger 200 towards the second end 103 of the barrel 101. In the example of FIG. 2, the plunger 200 includes a flange 205 to assist in manual manipulation of the plunger 200 relative to the syringe casing 100. Also in the example of FIG. 2, the plunger body 201 has a +-shaped cross-section along its length. However, the plunger 200 of FIG. 2 is presented here as an example only, and embodiments of the inventive concepts are not limited to the particularities of the plunger 200.

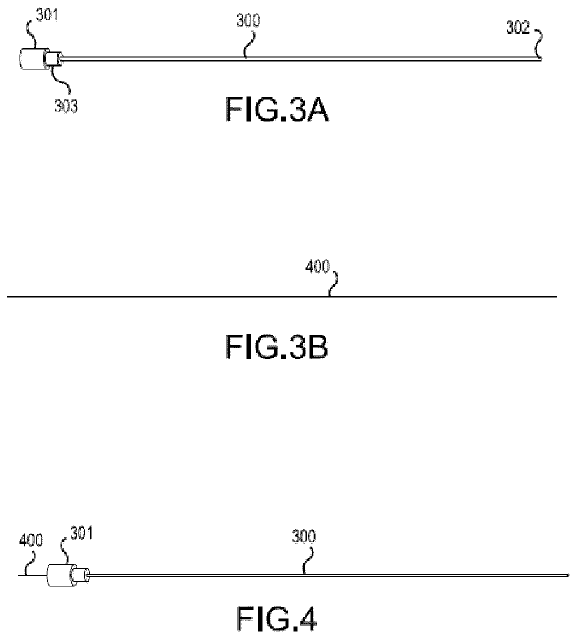

FIGS. 3A, 3B and 4 are perspective views for reference in describing examples of a hollow needle 300 and stylet 400. In particular, FIG. 3A is a perspective view of a hollow needle 300, FIG. 3B is a perspective view of a stylet 400, and FIG. 4 is a perspective view of the hollow needle 300 and stylet 400 in their assembled state. Referring collectively to these figures, a distal end 302 of the needle 300 may be configured for piercing tissue, and a proximal end 303 of the hollow needle 300 may be coupled to a hub 301. The hub 301 is configured for attachment to the hub 104 of the syringe casing 100. When attached, a lumen of the hollow needle 300 is in fluid communication with the aspiration cavity 106 of the syringe casing 100. The stylet 400 is configured for insertion through the hollow needle 300. The stylet 400 is intended to prevent or inhibit non-target cells from blocking the hollow needle 300 during insertion into tissue. In the assembled state (FIG. 4), the stylet 400 has a distal portion extending to the distal end 302 of the hollow needle 302 and a proximal portion extending within the syringe casing 100 (FIG. 1). As will be explained later, an extraction mechanism is located within the syringe casing 100 and is coupled to the proximal end of the stylet 400.

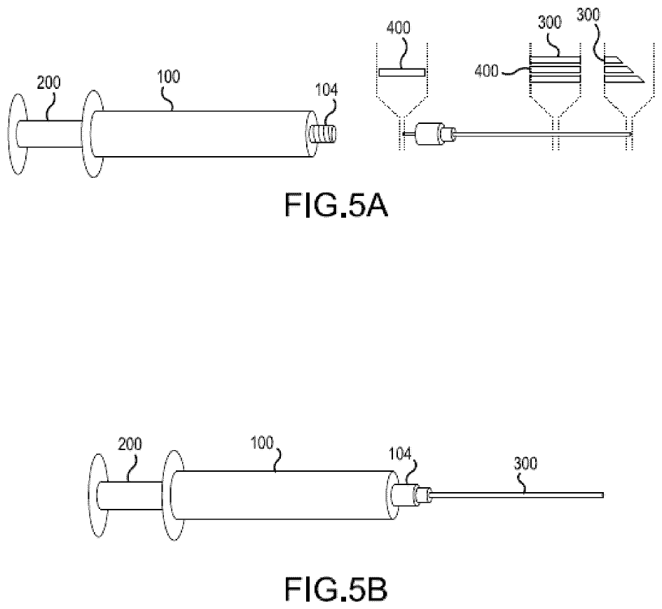

FIGS. 5A and 5B are perspective views for reference in describing an assembled syringe assembly according to example embodiments of the inventive concepts. In particular, FIG. 5A is a perspective view of the hollow needle 300/stylet 400 separated from the syringe casing 100. Although FIG. 5A illustrates the distal end of the stylet 400 aligned at a slant with the distal end of the hollow needle 300, the embodiments of the inventive concepts are not limited in this manner.

FIG. 5B is a perspective view of the syringe assembly with the needle 300/stylet 400 attached to the syringe casing 100. In the embodiment of FIG. 5B, the stylet 400 is not externally exposed. On other embodiments, however, a distal end of the stylet 400 may protrude slightly past a distal end of the hollow needle 300.

Although not visible in FIG. 5B, the proximal end of the stylet 400 is attached to an extraction mechanism located within the syringe assembly. In some embodiments of the inventive concepts, the extraction mechanism is spring-loaded and activated by a trigger accessible to the operator. Also, in some embodiments of the inventive concepts, the extraction mechanism is located in the plunger 200. In other embodiments of the inventive concepts, the extraction mechanism is located in the syringe casing 100.

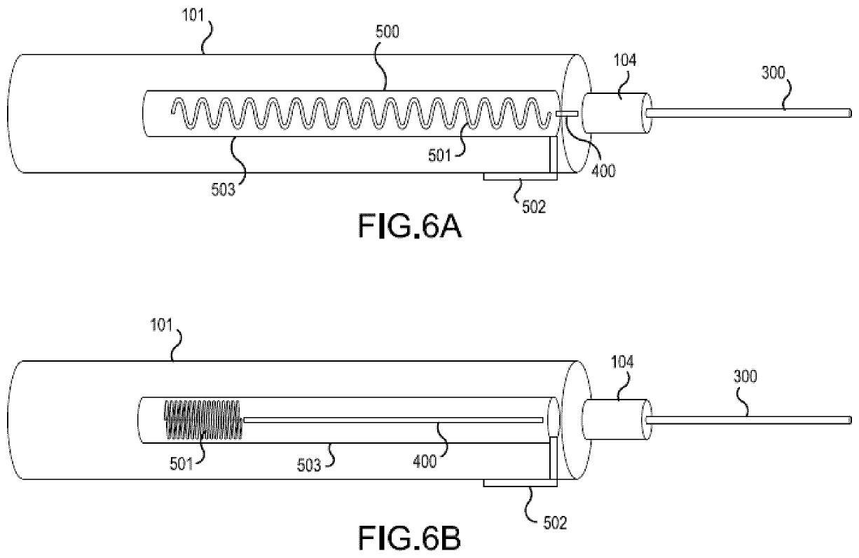

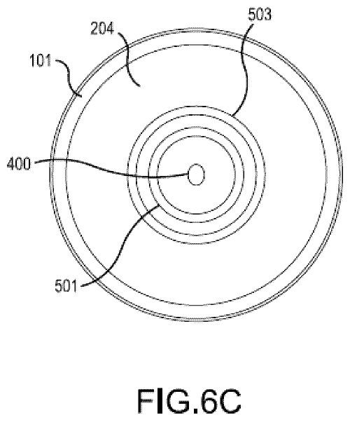

FIGS. 6A and 6B are perspective views for reference in describing an embodiment in which the extraction mechanism 500 is located in the syringe casing 100. In this embodiment, extraction mechanism 500 includes an extraction chamber 503 that is fixed to one or more inside walls of the syringe casing 101. The extraction chamber 503 may be tubular to conform to the shape of an extension spring 501 contained therein, and may extend coaxially within the barrel 101 of the syringe casing 100. The manner in which the extraction chamber 503 is fixed to the inner wall(s) of the syringe casing 100 can be varied by design, so long as fluid communication is not blocked between the hollow needle outlet of the hub 104 and the aspiration cavity 106 of the syringe casing 100.

FIG. 6A represents a state in which the stylet 400 extends through the hollow needle 300 (i.e., a state in which the hollow needle 300 is inserted into tissue). As shown, a proximal end portion of the stylet 400 is connected to one end portion of an extension spring 501 contained within the extraction chamber 503. The other end portion of the extension spring 501 is fixed in place at an opposite end of the extraction chamber 503. In this embodiment, the extension spring 501 is stretched along its length to generate a tensile load and held in place by a lever or latch or other mechanism. FIG. 6B represents a state in which the tensile load of the extension spring 501 is released by depressing a trigger (or button) 502 which detaches the extension spring 501 from the lever or latch or other mechanism that was holding the spring in place as in FIG. 6A. As a result, the extension spring 501 retreats to its non-loaded state, drawing the stylet 400 into the extraction chamber 503.

The plunger 200, which is not shown in FIGS. 6A and 6B, may include an aperture or opening along its length to allow it to travel along the length of the extraction chamber 503. In this case, referring to the cross-sectional view of FIG. 6C, the plunger seal 204 may have an outer circumferential sealing surface in contact with an inner wall of the barrel 101 of the syringe casing 100, and an inner circumferential sealing surface in contact with an outer wall of extraction chamber 503. Also represented in FIG. 6C is the spring 501 and stylet 400 contained with the extraction chamber 503.

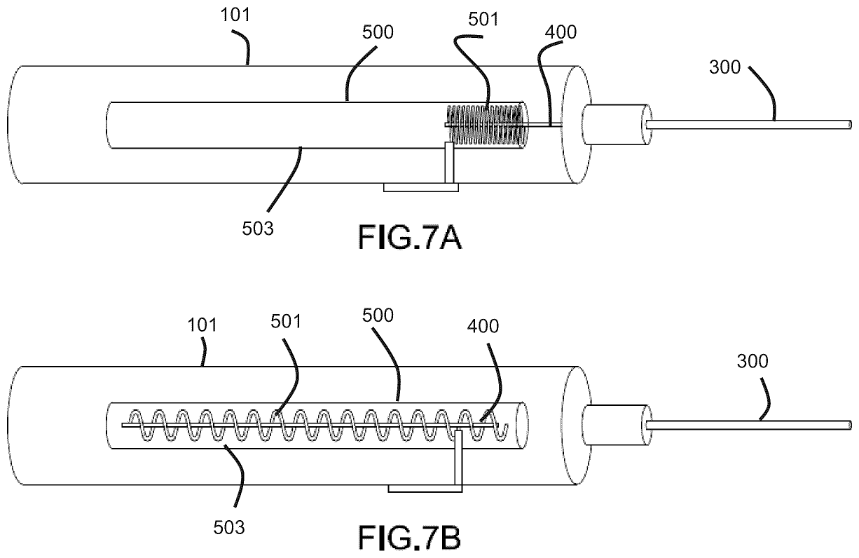

FIGS. 7A and 7B are perspective views for reference in describing another embodiment in which the extraction mechanism 500 is located in the syringe casing 101. This embodiment is the same as that of FIGS. 6A and 6B, except that the spring 501 is a compression spring instead of an extension spring. As such, as shown in FIG. 7A, the spring 501 is compressed in its initial state with the stylet 400 extending through the needle 300. A resultant compressive load is released by depressing the trigger 502, causing the spring 501 to expand to its non-loaded state, thus drawing the stylet 400 into the extraction chamber 503.

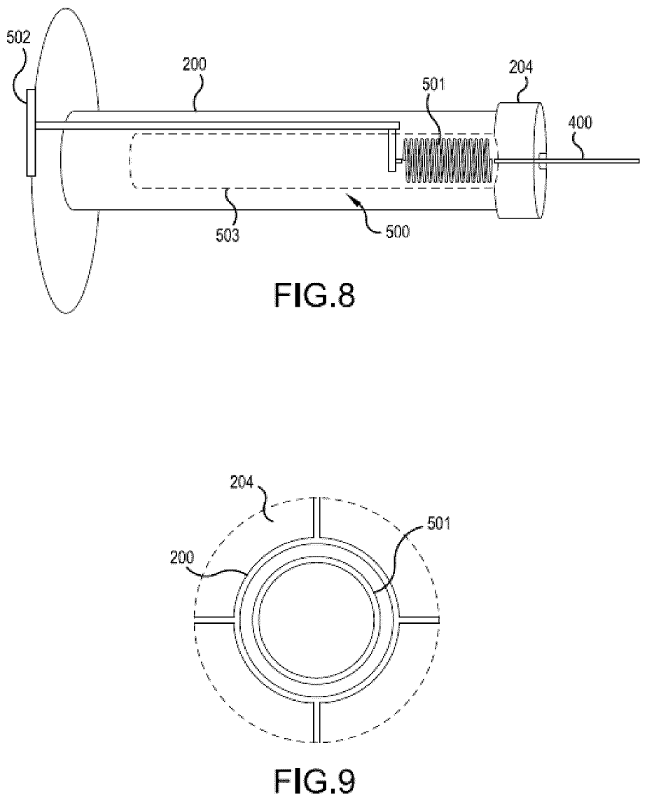

As mentioned above, in some embodiments of the inventive concepts, the extraction mechanism 500 is located in the plunger 200. An example of this is represented in FIG. 8 of the drawings. As shown, the extraction mechanism 500 may be embedded within the plunger 200. FIG. 8 depicts an example in which the extraction mechanism 500 includes an extraction chamber 503 and a compression spring 501 like that described above in connection with FIGS. 7A and 7B. In an alternative embodiment, the spring 501 may be an expansion spring like that of FIGS. 6A and 6B. In either case, a trigger 502 is located on the plunger to release a load (compression or expansion) of the spring 501 and draw the stylet 400 into the casing 503. Although not shown in FIG. 8, the plunger 200 is inserted in the syringe casing 101 with the stylet 400 extending through the hub 104 into the hollow needle 300.

In some embodiments, a separate chamber 503 is omitted. For example, referring to the cross-sectional view of FIG. 9, the inner walls of the plunger 200 may house the spring 501. In other words, a chamber of the plunger 200 may function as the extraction chamber. FIG. 9 also depicts a representation of the plunger seal 204 and ribs which may extend lengthwise along the outer diameter of the plunger 200. However, the inventive concepts are not limited to such a configuration.

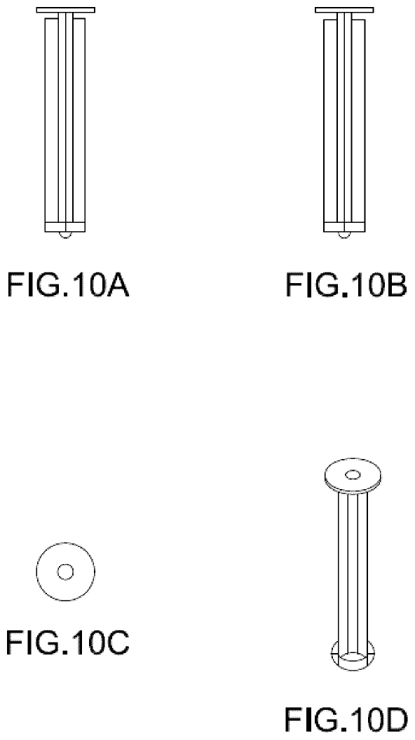

FIGS. 10A, 10B, 10C and 10D are additional perspective views of the plunger 200 including a stylet extraction mechanism. In particular, FIGS. 10A and 10B are side-views, FIG. 10C is a bottom view, and FIG. 10D is a perspective view. In this illustrated example, a flange is located at the top of the plunger 200, a needle/stylet attachment surface is located at the bottom of the plunger 200, and a stylet extraction chamber is located there between.

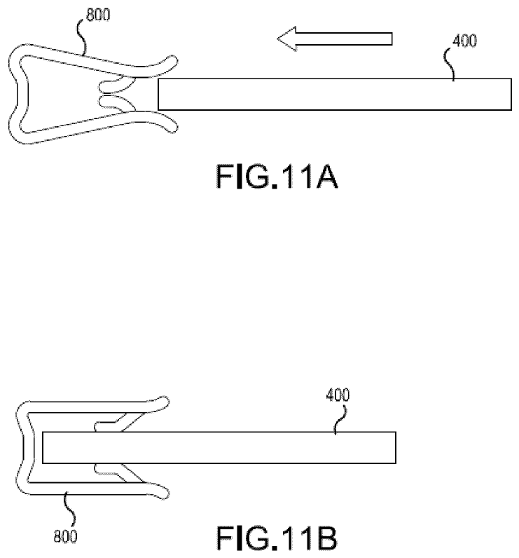

In some embodiments of the inventive concepts, the syringe assembly is prepackaged in a fully assembled state prior to use. In other embodiments of the inventive concepts, the needle 300/stylet 400 are attached to the syringe assembly by the operator (medical professional). Referring to FIG. 5A, this may be done by the operator inserting the stylet 400 into the proximal end of the syringe casing 100. The extraction mechanism of the syringe may be configured with a locking mechanism (operatively coupled to the extraction spring) configured to receive and lock-in-place the proximal end of the stylet 400. One example of such a locking mechanism is a ribbed channel configured to allow to stylet 400 to be inserted therein, but with sharp angular ridges or teeth configured to inhibit the stylet 400 from being detached after insertion. Another example of a locking mechanism is illustrated in FIGS. 11A and 11B. Here, a spring clamp 800 is configured to expand as the proximal end of the stylet 400 is pushed into the clamp 800. The style 400 remains seated in the clamp 800 primarily due to the clamping spring action of the clamp 800. If desired, one or more surfaces of the spring clamp 800 in contact with the stylet 400 can be roughened or ribbed to increase the frictional forces acting on the stylet 400. Further, although not shown, guide members can be included to direct the proximal end of stylet 400 into the spring clamp 800 (or other locking mechanism) when inserted into place. The inventive concepts are not limited by the particularities of the locking mechanism.

The embodiments described above utilize the stored energy of a compression or expansion spring or coil to extract the stylet from hollow needle. The inventive concepts, however, are not limited in this manner. For example, other types of springs may be utilized to draw the stylet from the hollow needle, such as torsion spring. Further, in addition to or in place of mechanical extraction mechanisms, electrical and/or magnetic mechanisms may be utilized to extract the stylet. In other words, any operator-triggered mechanism capable of storing the energy necessary to extract the stylet from the hollow needle may be utilized.

The location of the trigger (or button) used to activate the extraction mechanism is a matter of design. Ergonomically, it may be advantageous to place the trigger at a particular location, such as close to the needle-end of the syringe casing or at the push surface of the plunger. However, the inventive concepts are not limited to the location of the trigger.

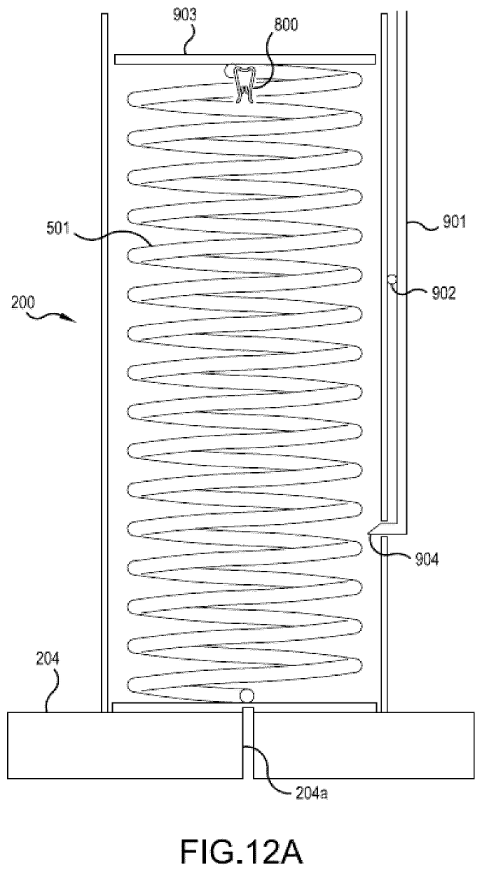

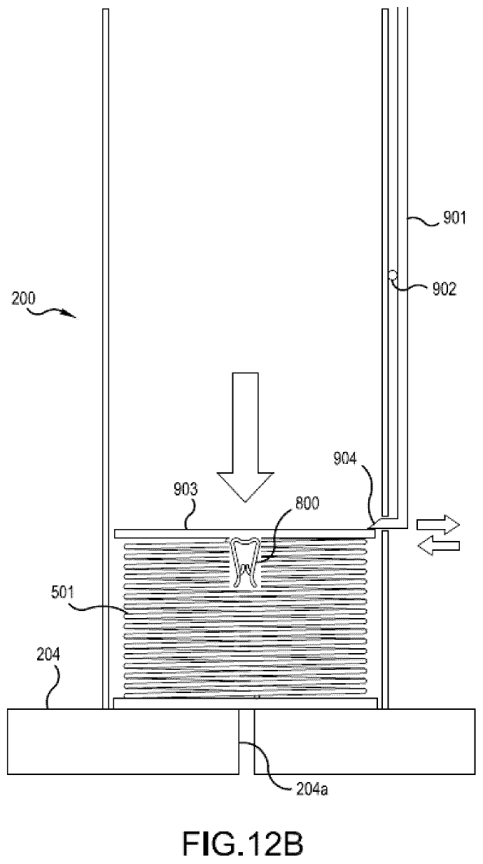

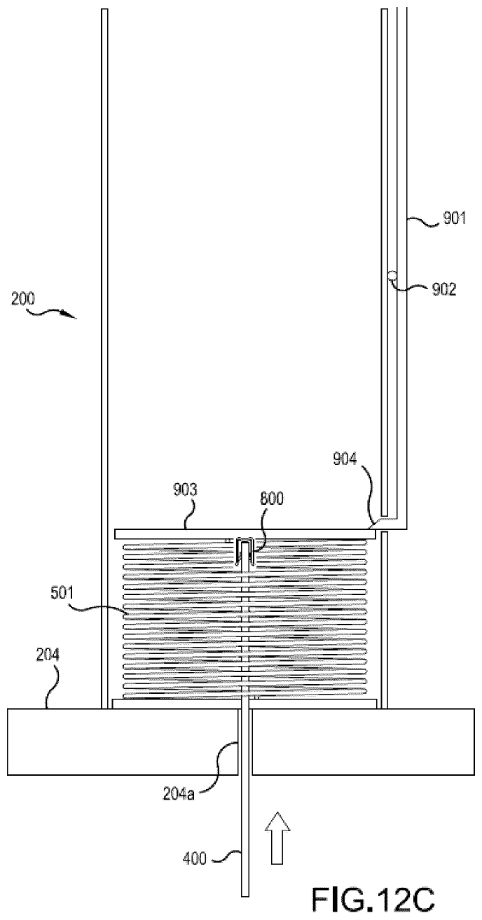

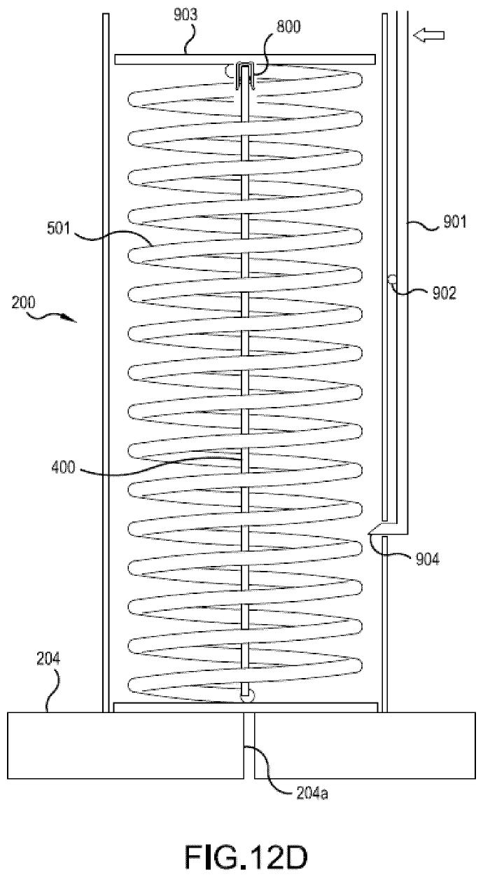

The inventive concepts are also not limited by the configuration of the trigger, and as those skilled in the art will appreciate, there are any number of mechanisms that can be conceived to release the potential energy of the spring fixed to the stylet. Nonetheless, FIGS. 12A, 12B, 12C and 12D illustrate a simple example in which a trigger bar 901 extends along the length of the plunger 200 and is pivotably mounted at a pivoting element 902 located on an outer sidewall of the plunger 200. FIG. 12A illustrates a “pre-loaded” state in which the expansion spring 501 is in its expanded and relaxed state, and the stylet is not yet engaged. As shown in FIG. 12B, the expansion spring 501 is set to a locked position by pressing an upper surface of a bar or plate 903 having a lower surface fixed to the spring 501. The bar or plate 903 forces an angled surface of the locking element 904 (and the trigger bar 901) outwardly as it is pushes pass the locking element 904, after which the locking element 904 returns to is initial position to lock the spring 501 in place as represented in FIG. 12B. The return of the locking element 904 to its initial position can be carried out manually by the operator, or automatically resulting from a spring action of the trigger bar 901 and/or pivot element. It is also noted that the expansion spring 501 can be set to its locked position either at the assembly factory prior to shipment, or by the operator prior to the FNA procedure. Next, as represented in FIG. 12C, the stylet 400 may be inserted through a central aperture 204a in the plunger seal 204 and then into a spring clamp 800 fixed to a lower surface of the bar or plate 903 attached at the one end of the spring 501. In this state, the extraction mechanism of the syringe assembly is ready for the FNA procedure. FIG. 12D represents a state in which the trigger mechanism has been activated by pressing the other end of the trigger bar 901. Pushing the other end of the trigger bar 901 causes the locking element 904 to withdraw from the plunger 200, thus releasing (or unlocking) the spring 501. As a result, the spring 501 expands as represented in FIG. 12D to extract the stylet 400 into the plunger 200. In this state, the plunger 200 may have a cross-section such as that shown in previously described FIG. 9.

As explained previously, in conventional arrangements, the hollow needle and the syringe are initially separated from one another at the start of the FNA procedure, and only later is the syringe attached to hollow needle during the FNA procedure (i.e., after extraction of the stylet). On the other hand, in some example embodiments of the inventive concepts, the hollow needle is attached in advance to the syringe. For example, the syringe assembly with the attached hollow needle may be pre-packaged as a single ready-for-use unit. That is, the syringe may be fully assembled and packaged inside a box or other enclosure. In this case, a removable sheath may cover the otherwise exposed hollow needle as a safety precaution. In practice, the operator would remove the syringe assembly from the package, remove the sheath (if any) covering the needle, and proceed with the FNA procedure. Other than removal of the sheath, the syringe assembly is ready for use when removed from the package. In this case, the extraction mechanism may be locked in a loaded position with the stylet extending through the hollow needle. Alternatively, the operator may lock the extraction mechanism in its loaded position after removing the syringe assembly from the package. Also, in some embodiments of the inventive concepts, the syringe assembly is a one-time use device that is disposed of after the FNA procedure.

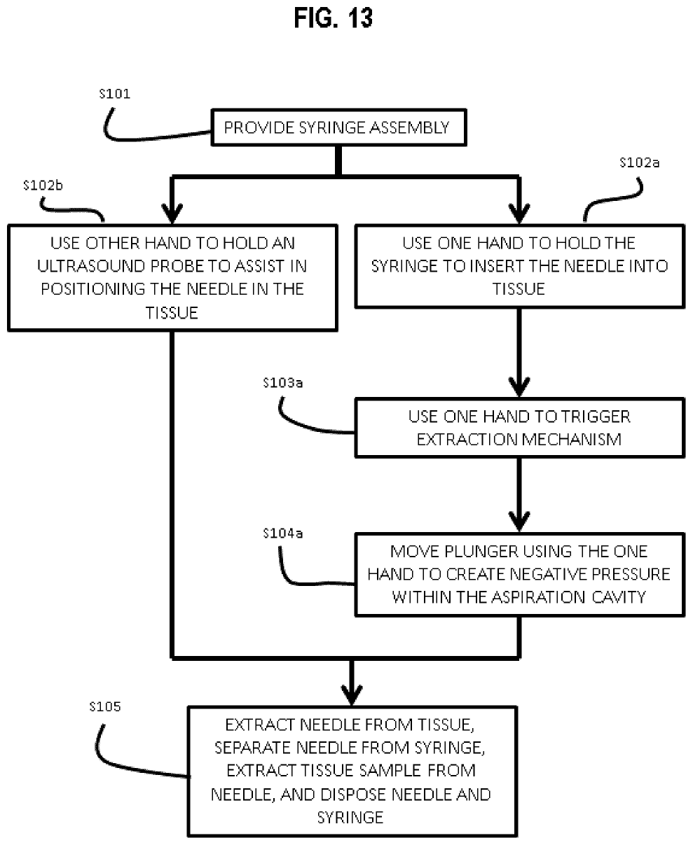

The inventive concepts include a FNA method such as that represented by the flowchart of FIG. 13. Referring to FIG. 13, the FNA method includes providing a syringe assembly (S101) according to one or more of the embodiments described above. The method further includes using one hand to hold the syringe to insert the needle into tissue (S102a) while using another hand to hold an ultrasound probe to assist in positioning the needle in the tissue (S102b). The method further includes triggering the button using the one hand (S103a) to cause the extraction mechanism to fully extract the stylet from the needle into the extraction chamber of the syringe while continuing to hold the ultrasound probe in the other hand, and moving the plunger using the one hand (S104a) to create negative pressure within the aspiration cavity while continuing to hold the ultrasound probe in the other hand, wherein the negative pressure causes tissue sample to be drawn into the needle. The method further includes extracting the needle from the tissue, separating the needle from the syringe, extracting the tissue sample from the needle and/or syringe, and optionally disposing of the needle and syringe (S105). That is, in some embodiments of the inventive concept, the syringe assembly is a single-use device that is disposed of after the FNA specimen is obtained.

As noted previously, the drawings are not necessarily drawing to scale. On a related note, the embodiments described herein are applicable to various needle gauges and lengths, as well as various syringe lengths and volumes. Different sizes/lengths of the syringe assembly parts and volumes may be preferred depending on tissue types and lesion locations. Further, in alternative embodiments, the extraction chamber and the stylet may be longer than the barrel of the syringe and/or longer than the plunger (i.e., the extraction chamber may extend past the tops of the syringe and/or plunger). Still further, in other alternative embodiments, the hollow needle is longer than the barrel of the syringe, but the extraction chamber (and stylet when extracted) is fully contained within the barrel of the syringe or within the plunger. In this case, for example, a monofilament may be attached on one end to a stylet within the hollow needle and on the other to a small cylinder in the extraction chamber. That cylinder may be locked in place and attached to a loaded spring that when unlocked results in rotation of the cylinder, wrapping the monofilament and retracting the stylet. This allows for the use of a stylet that is shorter than the hollow needle.

While embodiments of the inventive concepts have been described above with reference to drawings, it will be apparent to those of ordinary skill in the art that various changes and modifications may be made thereto without departing from the spirit and scope of the inventive concepts.

What is claimed is:

1. A pre-packaged syringe assembly, comprising:
a syringe assembly; and
an outer package containing the syringe assembly,
wherein the syringe assembly comprises:
a syringe casing defining an aspiration cavity therein and including a barrel having opposite first and second ends;
a plunger movable within the barrel to define a volume of the aspiration cavity;
a hollow needle having a distal end configured for piercing tissue, and a proximal end detachably fixed to the first end of the syringe casing, the hollow needle in fluid communication with the aspiration cavity;
a stylet having a distal portion extending within the needle and a proximal portion extending within the syringe casing;
an extraction mechanism located within the syringe casing and coupled to the proximal portion of the stylet; and
a trigger at least partially exposed externally of the aspiration cavity of the syringe casing, wherein the trigger when activated causes the extraction mechanism to extract an entirety of the stylet from the needle to within the syringe casing.

2. The pre-package syringe assembly of claim 1, further comprising a protective sheath covering the hollow needle within the outer package.

3. The pre-packaged syringe assembly of claim 1, wherein a distal end of the stylet extends past a distal end of the hollow needle within the outer package.

4. The pre-packaged syringe assembly of claim 2, wherein the protective sheath further covers a distal end of the stylet that extends past a distal end of the hollow needle within the outer package.

5. The pre-packaged syringe assembly of claim 1, wherein the syringe assembly is a single-use disposable fine needle aspiration (FNA) syringe assembly.

6. The syringe assembly of claim 1, wherein the extraction mechanism is configured to store potential energy while the syringe assembly is contained within the outer package, and upon removal of the syringe assembly from the outer package, the trigger is configured to release the stored potential energy of the extraction mechanism to extract the stylet from the hollow needle into the syringe.

7. The syringe assembly of claim 6, wherein the potential energy is at least one of mechanical or electrical or magnetic energy.

8. The syringe assembly of claim 6, wherein the extraction mechanism is located within the barrel of the syringe casing.

9. The syringe assembly of claim 6, wherein the extraction mechanism is located within the plunger.

10. The syringe assembly of claim 6, wherein the extraction mechanism includes an extraction chamber extending coaxially within the barrel of the syringe casing, and the extraction chamber is configured to house the stylet when extracted from the hollow needle into the extraction chamber.

11. The syringe assembly of claim 10, wherein the extraction mechanism is a spring-loaded device and further includes a spring located within the extraction chamber and coupled to the proximal portion of the stylet, and
wherein the spring is configured to extract the stylet from the hollow needle into the extraction chamber when the trigger is activated.

12. The syringe assembly of claim 11, wherein the spring is helical and extends coaxially within the extraction chamber, and
wherein the trigger when activated releases a stored potential energy of the spring to draw the stylet into the extraction chamber.

13. The syringe assembly of claim 12, wherein the spring is an extension spring.

14. The syringe assembly of claim 12, wherein the spring is a compression spring.

15. The syringe assembly of claim 10, wherein the plunger extends around and is movable along a length of the extraction chamber, the plunger includes a plunger seal extending around the extraction chamber, and
wherein the plunger seal includes an inner surface in contact with an outer wall of the extraction chamber, and an outer surface in contact with an inner wall of the barrel of the syringe casing.

16. The syringe assembly of claim 6, wherein the extraction mechanism is located within the plunger, and
the plunger is hollow to define an extraction chamber therein, the extraction chamber configured to house the stylet when extracted from the hollow needle.

17. The syringe assembly of claim 16, wherein the extraction mechanism is a spring-loaded device and further includes a spring located within the extraction chamber of the plunger and coupled to the proximal portion of the stylet, and
wherein the spring is configured to extract the stylet from the hollow needle into the extraction chamber of the plunger when the trigger is activated.

18. The syringe assembly of claim 17, wherein the spring is helical and extends coaxially within the extraction chamber of the plunger, and
wherein the trigger when activated releases a stored potential energy of the spring to draw the stylet into the syringe.

19. The syringe assembly of claim 18, wherein spring is either one of an extension spring or a compression spring.

20. The syringe assembly of claim 19, wherein the plunger includes a plunger seal having an opening therein, wherein the stylet extends through the opening in the plunger seal.
